(19) United States
(12) Patent Application Publication
(10) Pub. No.: US 2019/0086326 A1
(43) Pub. Date: Mar. 21, 2019

(54) MEDICINE MONITORING DEVICE, SYSTEM AND METHOD

(71) Applicant: BOE TECHNOLOGY GROUP CO., LTD., BEIJING (CN)

(72) Inventor: Kailiang ZHANG, Beijing (CN)

(21) Appl. No.: 16/087,378

(22) PCT Filed: Mar. 2, 2018
(86) PCT No.: PCT/CN2018/077896

§ 371 Date: Sep. 21, 2018

(30) Foreign Application Priority Data
Mar. 16, 2017 (CN) 201710157533.4

(51) Int. Cl. G01N 21/31; G16H 20/10; A61J 1/03; A61J 7/00; A61J 7/02

(52) U.S. Cl. CPC G01N 21/31; G16H 20/10; G16H 80/00; A61J 7/0076; A61J 7/02; A61J 1/03

(57) ABSTRACT

The present application provides a medicine monitoring device, system and method. The medicine monitoring device comprises: a medicine analyzer configured to detect a type parameter and an amount parameter of a medicine; and a processing module circuit coupled to the medicine analyzer. The processing module circuit is configured to determine a detected type and amount of the medicine based on the detected type parameter and amount parameter of the medicine, obtain preset type and amount of the medicine, and judge whether the detected type and amount of the medicine coincide with the preset type and amount of the medicine.

### CROSS REFERENCE TO RELATED APPLICATIONS

The present application is a U.S. National Stage Application under 35 U.S.C. § 371 of International Application No. PCT/CN2018/077896, filed on Mar. 2, 2018, which claims priority to Chinese Patent Application No. 201710157533.4 filed on Mar. 16, 2017, the disclosure of which is incorporated herein in its entirety by reference.

### TECHNICAL FIELD

The present application relates to a medicine monitoring device, system and method.

### BACKGROUND

In contemporary society, with the increasing stress in life, the number of people suffering from chronic diseases such as physical or mental diseases continuously grows, and it is often necessary for those people to suppress the diseases by taking medicines for a long time. However, a considerable number of middle and young people often forget to take medicine because of the fast pace of life and busy work. In addition, for people with serious illness, there may be five or six or even dozen medicines to be taken a day, and each needing to be taken in different amounts, or by different times, which makes it difficult for the patients to remember whether they have taken the medicines. Furthermore, many medicines are named after chemical composition or transliteration. For many patients, those clumsy names are difficult to remember clearly and correctly, which may lead to taking the wrong medicines. For aged people, most of them have presbyopia eyes and cannot identify the medicines' names most often. For smaller tablets, it is even difficult for the aged people to count the number of tablets.

### SUMMARY

According to some embodiments of the present disclosure, a medicine monitoring device is provided, comprising: a medicine analyzer configured to detect a type parameter and an amount parameter of a medicine; and a processing circuit coupled to the medicine analyzer. The processing circuit is configured to: determine a detected type and amount of the medicine based on the detected type parameter and amount parameter of the medicine, obtain preset type and amount of the medicine, and judge whether the detected type and amount of the medicine coincide with the preset type and amount of the medicine.

According to some embodiments of the present disclosure, the medicine analyzer is configured to detect the type parameter and the amount parameter of the medicine placed in a medicine container.

According to some embodiments of the present disclosure, the medicine analyzer is configured to detect the type parameter and the amount parameter of the medicine taken out from a medicine container.

According to some embodiments of the present disclosure, the medicine analyzer comprises: a Raman analyzer comprising a laser device and a laser detecting device, wherein laser light emitted by the laser device is capable of being irradiated onto a surface of the medicine, and the laser detecting device is capable of receiving reflected laser light and obtaining a spectrum of the medicine; and a weight meter configured to detect a weight of the medicine in the medicine container.

According to some embodiments of the present disclosure, the medicine monitoring device further comprises the medicine container, wherein the medicine container comprises at least one subunit for holding the medicine, each subunit is provided with a through hole and is correspondingly provided with the laser device and the laser detecting device provided, the laser device being configured to emit the laser light to be irradiated onto the surface of the medicine in the subunit through the through hole, the laser detecting device being capable of receiving the laser light reflected back through the through hole and obtaining the spectrum of the medicine in the subunit; and each subunit is correspondingly provided with the weight meter configured to detect the weight of the medicine in the subunit.

According to some embodiments of the present disclosure, the medicine monitoring device further comprises a storage circuit in which a spectrum and a tablet weight of a plurality of medicines are stored in advance.

According to some embodiments of the present disclosure, the processing circuit is further configured to: determine a type of the medicine in the subunit based on matching between a detected spectrum of the medicine in each subunit and a spectrum of the medicine stored in advance; calculate an amount of the medicine in the subunit using the detected weight of the medicine in the subunit and the tablet weight of this type of medicine, thereby determining the type and the amount of the medicine placed in the medicine container.

According to some embodiments of the present disclosure, in a case where the medicine analyzer is configured to detect the type parameter and the amount parameter of the medicine taken out from the medicine container, the processing circuit is further configured to: determine a type of the medicine in the subunit based on matching between a spectrum of the medicine in each subunit which is detected before a take-out operation of a user and a spectrum of the medicine stored in advance; calculate an amount of this type of medicine which has been taken out from the medicine container according to a weight of this type of medicine in the subunit which is detected before the take-out operation of the user, a weight of this type of medicine in the subunit which is detected after the take-out operation of the user and a tablet weight of this type of medicine, thereby determining the type and the amount of the medicine taken out from the medicine container.

According to some embodiments of the present disclosure, each subunit is configured to allow only one tablet of medicine to be placed, and the processing circuit is further configured to: determine, in response to detecting a weight of the medicine in the subunit to be zero after the take-out operation of the user, the type and the amount of the medicine taken out from the subunit according to a type of the medicine in the subunit which is detected before the take-out operation of the user, thereby determining the type and the amount of the medicine taken out from the medicine container.

According to some embodiments of the present disclosure, the processing circuit is configured to output a prompting signal in response to judging that the detected type and amount of the medicine do not coincide with the preset type and amount of the medicine.

According to some embodiments of the present disclosure, the medicine monitoring device further comprises: a prompting circuit coupled to the processing circuit and configured to present a prompt to a user based on the prompting signal output by the processing circuit.

According to some embodiments of the present disclosure, the medicine monitoring device further comprises a communication circuit coupled to the processing circuit and configured to: transmit the prompting signal output by the processing circuit to an electronic device in communication with the medicine monitoring device, receive medication information transmitted from the electronic device, wherein the medication information comprises at least medication time, a type and an amount of the medicine to be taken.

According to some embodiments of the present disclosure, there is provided a medicine monitoring system, comprising the medicine monitoring device as described above and an electronic device in communication with the medicine monitoring device, wherein the medicine monitoring device interacts with the electronic device via the communication device.

According to some embodiments of the present disclosure, there is provided a medicine monitoring method, comprising the steps of: determining a type and an amount of a medicine placed in a medicine container; judging whether the determined type and amount of the medicine placed in the medicine container coincide with a preset type and amount of the medicine; outputting a first prompt in response to judging that the determined type and amount of the medicine placed in the medicine container do not coincide with the preset type and amount of the medicine.

According to some embodiments of the present disclosure, the medicine monitoring method further comprises: outputting a second prompt in response to determining that more than two types of medicines are contained in each of the at least one subunit of the medicine container.

According to some embodiments of the present disclosure, there is also provided a medicine monitoring method, comprising: determining a type and an amount of a medicine taken out from a medicine container; judging whether the determined type and amount of the medicine taken out from the medicine container coincide with a preset type and amount of the medicine; outputting a first prompt in response to judging that the determined type and amount of the medicine taken out from the medicine container do not coincide with the preset type and amount of the medicine.

According to some embodiments of the present disclosure, the medicine monitoring method further comprises: outputting a second prompt in response to determining that more than two types of medicines are contained in each of the at least one subunit of the medicine container.

According to some embodiments of the present disclosure, the medicine monitoring method further comprises: outputting a third prompt information in response to determining that no medicine is taken out from the medicine container.

### BRIEF DESCRIPTION OF THE DRAWINGS

FIG. 1 is an exemplary structural block diagram of a medicine monitoring device in accordance with some embodiments of the present disclosure;

FIG. 2 is an exemplary structural block diagram of a medicine monitoring device in accordance with some embodiments of the present disclosure;

FIG. 3 is an exemplary structural block diagram of a medicine monitoring system in accordance with some embodiments of the present disclosure;

FIG. 4 is a top plan view of an exemplary structure of a medicine container of a medicine monitoring device in accordance with some embodiments of the present disclosure;

FIG. 5 is a schematic flow diagram of a medicine monitoring method in accordance with some embodiments of the present disclosure;

FIG. 6 is a schematic flow diagram of a medicine monitoring method in accordance with some embodiments of the present disclosure;

FIG. 7 is a schematic flow diagram of a medicine monitoring method in accordance with some embodiments of the present disclosure.

### DETAILED DESCRIPTION

In order to make the above objects, features, and advantages of the present disclosure more comprehensible, the present disclosure will be further described in detail with reference to the accompanying drawings and specific embodiments.

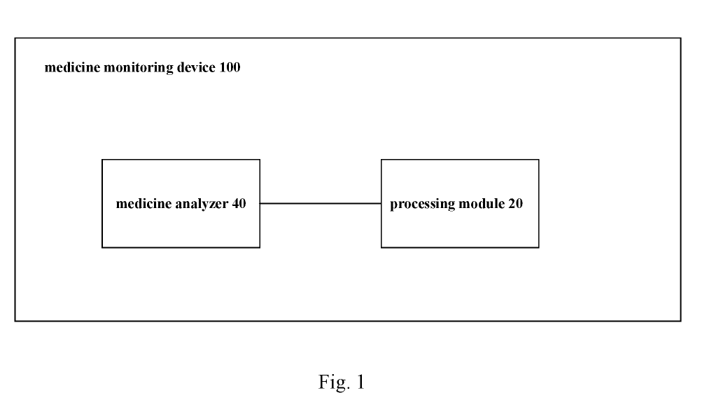

According to some embodiments of the present disclosure, a medicine monitoring device is provided. FIG. 1 is an exemplary structural block diagram of a medicine monitoring device 100 in accordance with some embodiments of the present disclosure. As shown in FIG. 1, the device 100 comprises a medicine analyzer 40 and a processing circuit 20. The medicine analyzer 40 is capable of detecting a type parameter and an amount parameter of a medicine. The processing circuit 20 is coupled to the medicine analyzer 40 and is capable of determining a detected type and amount of the medicine based on the detected type parameter and amount parameter of the medicine, obtaining preset type and amount of the medicine, and judging whether the detected type and amount of the medicine coincide with the preset type and amount of the medicine.

In some embodiments, the medicine analyzer 40 can detect a type parameter and an amount parameter of a medicine placed in a medicine container (not shown in FIG. 1). The processing circuit 20 can determine a type and an amount of the medicine placed in the medicine container according to the type parameter and the amount parameter of the medicine placed in the medicine container (not shown in FIG. 1) detected by the medicine analyzer 40, and judging whether the type and the amount of the medicine placed in the medicine container coincide with preset type and amount of the medicine.

In other embodiments, the medicine analyzer 40 can detect a type parameter and an amount parameter of a medicine taken out from the medicine container. Here, for example, it can be determined by detecting a type parameter and an amount parameter of the original medicine in the medicine container and a type parameter and an amount parameter of the medicine in the medicine container after a take-out operation and comparing these parameters. The processing circuit 20 can determine the type and the amount of the medicine taken out from the medicine container according to the type parameter and the amount parameter of the medicine taken out from the medicine container detected by the medicine analyzer 40, and judge whether the type and the amount of the medicine taken out from the medicine container coincide with preset type and amount of the medicine.

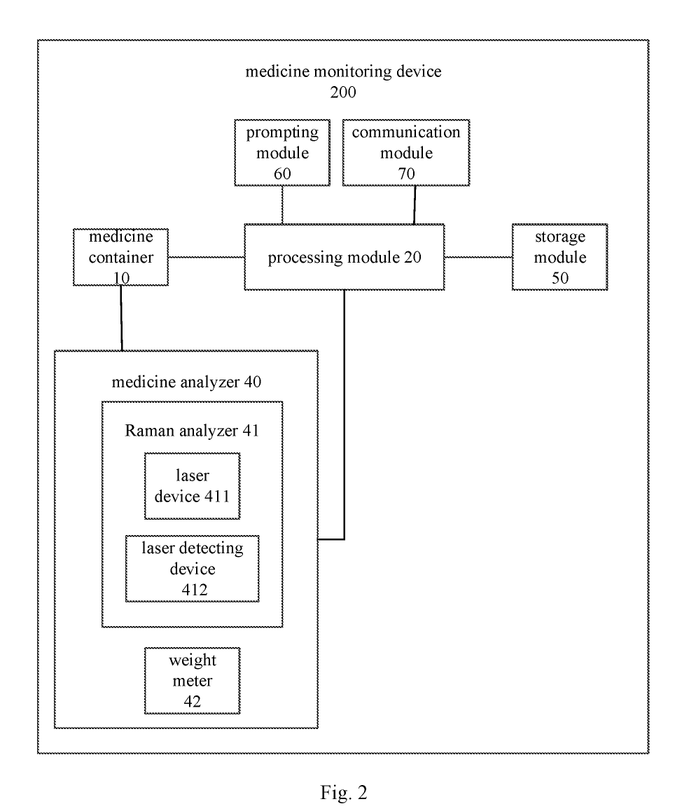

FIG. 2 shows an exemplary structural block diagram of a medicine monitoring device 200 in accordance with some embodiments of the present disclosure. As shown in FIG. 2, similar to FIG. 1, the medicine monitoring device 200 comprises a processing circuit 20 and a medicine analyzer 40.

The medicine analyzer 40 may comprise, for example, a Raman analyzer 41 and a weight meter 42. The Raman analyzer 41 may comprise a laser device 411 and a laser detecting device 412. Laser light emitted by the laser device 411 can be irradiated onto a surface of the medicine. The laser detecting device 412 can receive laser light reflected back by, for example, a medicine in the medicine container 10 and obtain a spectrum of the medicine as a type parameter which is used by the processing circuit 20 to determine the type of the irradiated medicine. The weight meter 42 is used, for example, to collect the weight of the medicine in the medicine container 10 as an amount parameter which is used by the processing circuit 20 to determine the amount of the irradiated medicine.

The processing circuit 20 can obtain the type parameter and the amount parameter of the medicine detected by the Raman analyzer 41 and the weight meter 42 of the medicine analyzer 40, compare the obtained type parameter and amount parameter of the medicine with a preset type parameter (for example, a preset spectrum) and a preset amount parameter (a preset tablet weight of the medicine) to determine a detected type and amount of the medicine. The processing circuit 20 may further compare the determined and detected type and amount of the medicine with preset type and amount of the medicine to judge whether the detected type and amount of the medicine coincide with the preset type and amount of the medicine.

The medicine monitoring device 200 may further comprise a medicine container 10 for holding a medicine.

FIG. 4 shows a top plan view of an exemplary structure of a medicine container in accordance with some embodiments of the present disclosure. As shown in FIG. 4, the medicine container 10 may comprise at least one subunit 11 for holding a medicine. Each subunit 11 may be used, for example, to hold the same type of medicine, but is not limited thereto. Each subunit may be used to hold the different types of medicines.

Each subunit 11 is provided with a through hole 12 and is correspondingly provided with a laser device 411 and a laser detecting device 412, the laser device 411 being configured to emit laser light to be irradiated onto the surface of the medicine in the subunit 11 through the through hole 12, the laser detecting device 412 being capable of receiving laser light reflected back through the through hole 12 and obtaining a spectrum of the medicine in the subunit 11.

For example, the through hole 12 is provided at the bottom of the subunit 11. A plurality of laser devices 411 may be provided, each of which may be disposed corresponding to a subunit 11. The laser device 411 is used to emit laser light to be irradiated onto the surface of the medicine in the subunit 11 through the through hole 12. The through holes 12 respectively formed at the bottom of the respective subunits 11 may have a specific size. For example, the size of the through hole 12 can be set according to a size of an actual shape of a medicine, ensuring that the surface of the medicine can be irradiated with laser through the through hole 12 without dropping it from the through hole 12.

The processing circuit 20 can obtain a type of the medicine in each subunit 11 using, for example, a Raman analyzer 41 (for example, comprising a laser device 11 and a laser detecting device 412) by the following process. The laser device 411 located at the bottom of the subunit 11 emits laser light which is irradiated onto the medicine in the subunit 11 through the through hole 12 provided at the bottom of the subunit 11. For example, when there is only one tablet in the subunit 11, the laser hits the tablet. When there is a plurality of tablets in the subunit 11, the laser can hit any one of the tablets, that is, all the tablets can be hit by the laser. According to the principle of light reflection, reflection occurs after the laser hits the medicine, and the reflected light is received by the laser detecting device 412. Thus, the Raman analyzer 41 analyzes the reflected light using the Raman scattering principle to obtain a Raman spectrum of the medicine. The processing circuit 20 can compare the Raman spectrum obtained by the Raman analyzer 41 with a predetermined medicine spectrum library to determine a type of the medicine in the subunit 11. The predetermined medicine spectrum library may be obtained, for example, from an external database, or may be stored in advance, for example, in the storage circuit 50.

For a plurality of subunits, the types of medicines in the respective subunits 11 can be obtained simultaneously or sequentially.

Each subunit 11 may be correspondingly provided with a weight meter configured to detect a weight of the medicine in the subunit. That is, a plurality of weight meters 42 may be provided and each of the weight meters 42 may be provided corresponding to a subunit 11. The weight meter 42 collects the weight of the medicine in the subunit 11.

For example, the processing circuit 20 can obtain an amount of the medicine in each subunit 11 using the weight meter 42 by the following process. In some embodiments, the weight meter 42 detects the weight of the medicine in the subunit 11, and the processing circuit 20 utilizes the type of the medicine in the subunit 11 determined as described above, the weight of the medicine in the subunit 11 detected by the weight meter 42, and a predetermined table weight of this type of medicine to calculate an amount of the medicine in the subunit 11.

For example, the processing circuit 20 can obtain an amount of a medicine taken out from each subunit 11 using the weight meter 42 by the following process. In some embodiments, the weight meter 42 detects a weight of the medicine in the subunit 11 before a take-out operation and a weight of the medicine in the subunit 11 after the take-out operation. The processing circuit 20 utilizes the type of the medicine in the subunit 11 determined as described above, a difference between the weights of the medicine in the subunit 11 before and after the take-out operation detected by the weight meter 42, and a predetermined table weight of this type of medicine to calculate an amount of the medicine taken out from the subunit 11.

For example, in some embodiments, the processing circuit 20 first determines the types of the medicines in the respective subunits 11 using a medicine analyzer and collects weight information of the medicines (for example, total weights of the medicines) in the respective subunits 11 using the weight meter 42 on the premise that the types of the medicines have been known. Then, based on a predetermined tablet weight information of each medicine, an amount of each medicine is obtained by dividing the total weight of the medicine by the tablet weight.

In some embodiments, the processing circuit 20 may determine the type of a medicine in each subunit 11 based on matching between a spectrum of the medicine in the subunit 11 and a spectrum of the medicine which is stored in advance, calculate an amount of the medicine in the subunit 11 using the detected weight of the medicine in the subunit 11 and the tablet weight of this type of medicine, thereby determining the type and the amount of the medicine placed in the medicine container 10.

In some embodiments, the medicine analyzer is configured to detect a type parameter (e.g., a spectrum) and an amount parameter (e.g., a weight) of the medicine taken out from the medicine container 10, and the processing circuit 20 may be configured to determine the type of the medicine in each subunit 11 based on matching between the spectrum of the medicine in the subunit 11 detected before a take-out operation of a user and a spectrum of the medicine stored in advance, calculate a weight of this type of the taken out medicine according to a weight of this type of the medicine in the subunit 11 detected before the take-out operation of the user, a weight of this type of the medicine in the subunit detected after the take-out operation of the user, and a tablet weight of this type of the medicine, thereby determining the type and the amount of the medicine taken out from the medicine container 10.

In some embodiments, each subunit 11 is configured to hold only one tablet, and the weight of the medicine in the subunit 11 becomes to zero after the medicine is taken out from the subunit 11. In this case, the processing circuit 20 may be configured to determine, in response to detecting the weight of the medicine in the subunit 11 after the take-out operation of the user to be zero, a type and an amount (i.e., one tablet) of the medicine taken out from the subunit 11 according to the type of the medicine in the subunit 11 detected before the take-out operation of the user. The type and the amount of the medicine taken out from the medicine container 10 can be determined by counting the type and the amount of the medicine in each subunit in which the type of the medicine has been detected and the weight of the medicine is detected to be zero (for example, adding the amounts of the medicine of the same type).

In some embodiments, the processing circuit 20 may be configured to judge whether a medicine has been taken during a medication time period, i.e., whether the medicine has been taken out from the medicine container 10. For example, the processing circuit 20 judges whether the medicine in each subunit 11 has been taken out by using information about the total weight of the medicine in the subunit 11 detected by the weight meter 42 during the medication time period. For example, if the total weight of the medicine in the subunit 11 is decreased, it is determined that the medicine has been taken out from the subunit 11; otherwise, it is determined that the medicine has not been taken out.

As shown in FIG. 2, optionally, the medicine monitoring device 200 may further comprise a storage circuit 50. Spectrums and the tablet weights of a plurality of medicines can be stored in the storage circuit 50 in advance. The storage circuit 50 may store at least a medicine spectrum library, a weight database of medicine, and medication information, wherein the medication information comprises at least medication time, and types and amounts of medicines that should be taken at corresponding medication time.

As shown in FIG. 2, optionally, the medicine detecting device 200 may further comprise a prompting circuit 60 for issuing a specific prompt information.

For example, after the processing circuit 20 has obtained the types of medicines in the respective subunits 11, if it has been found that more than two types of medicines are contained in one or more subunits 11, the processing circuit 20 may use the prompting circuit 60 to issue a first prompt information (for example, an alarm or vibration is issued) to alert the patient that the medicines have been dispensed incorrectly, and ask the patient to re-dispense the medicines.

For example, when the processing circuit 20 judges that the medicines in the respective subunits 11 have not been taken out using the information about the total weights of the medicines in the subunits 11 detected by the weight meter 42 during the medication time period, the prompting circuit 60 can be utilized to issue a second prompt information, for example, to prompt the user to take medicine. The process can be set to be performed within a specific time period according to actual needs. For example, a detection is performed only during a time period in which medicines should be taken (for example, 8:00-8:30, 12:00-12:30, etc.). This particular time period can be stored, for example, in the storage circuit 50 as the medication information. In addition, the medication information may also comprise information such as types and amounts of medicines to be taken at corresponding medication time.

As another example, the processing circuit 20 can perform classification and statistics according to the determined types of the medicines, and obtain how many types of the medicines are there in the medicine container 10, the amount of each type of the medicine, and monitor a change in weight information of the medicine during a specific medication time period, so as to determine the types and the amounts of medicines taken by a patient and compare them with the medication information stored in the storage circuit 50 in advance to judge whether the correct medicines have been taken by the patient this time. If both the types and the amounts of the medicines are correct, no prompt may be issued, or the user may be prompted that the medicines can be taken. If one or both of the type and the amount information of the medicines is incorrect, the prompting circuit 60 may be used to issue a third prompt information to prompt the user to check the types and the amounts of the taken-out medicines.

As shown in FIG. 2, optionally, the medicine detecting device 200 may further comprise a communication circuit 70. For example, the communication circuit 70 can be coupled to the processing circuit 20 and in communication with an external electronic device. For example, the communication circuit 70 can transmit a prompt signal output by the processing circuit 20 to the electronic device in communication with the medicine monitoring device and receive the medication information transmitted from the electronic device, wherein the medication information comprises at least medication time, types and amounts of medicines to be taken.

It should be noted that each of the modules may be a soft module or a hardware module. When the above modules are software modules implemented by a computer program language, the software modules can be executed on an electronic device to implement corresponding functions. When the above modules are hardware modules implemented by hardware circuits, the function corresponding to each module can be realized by specific components.

Embodiments of the present disclosure have at least one or more of the following advantages over the related art.

Firstly, the spectral characteristics of a medicine in the container can be identified by a Raman analyzer according to different spectrums and can be compared with a medicine spectrum library prepared in advance to obtain the types of the medicines, thereby avoiding the problem of misuse of medicines due to incorrect dispensing of medicines or taking out the wrong medicines.

Secondly, it is also possible to use the weight meter to detect weight information of the medicines in the subunits, remind the patient to take medicines during a medication time period, and ensure that the amounts of the medicines to be taken are consistent with the preset amounts.

With the medicine monitoring device provided by the embodiment of the present disclosure, it can avoid misuse of medicines by the patient caused by placing tablets of different kinds into the same subunit when the tablets are placed in the subunits and can avoid misuse of medicines by the patient caused by taking out incorrect medicines or wrong amounts of the medicines when taking the medicines.

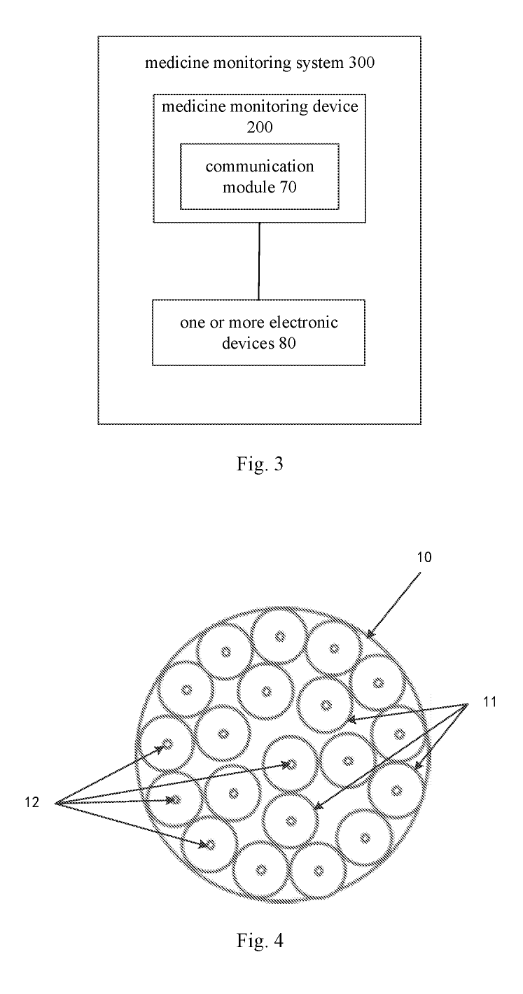

FIG. 3 shows an exemplary structural block diagram of a medicine monitoring system 300 in accordance with some embodiments of the present disclosure. As shown in FIG. 3, the medicine monitoring system 300 can comprise a medicine monitoring device 200, and one or more electronic devices (e.g., mobile terminals) 80. The electronic devices comprise, but are not limited to, smart electronic devices. The electronic devices comprise, but are not limited to, mobile phones, iPads, tablets, and the like.

If there is only one electronic device 80, the user can be the patient himself or herself If there are a plurality of electronic devices 80 involved, the users may comprise the patient's family members, friends, caregivers, and the like, in addition to the patient himself or herself. In the second case, the way of issuing the first prompt information may further comprise, for example, sending an information from a second electronic device (the mobile phone of a family member) to a first electronic device (the mobile phone of the patient), prompting that the medicines have been dispensed incorrectly and asking the patient to check and re-dispense.

With the communication circuit 70, the medicine monitoring device 200 and the electronic device 80 can interact by wireless transmission (mobile Internet, Bluetooth, etc.). The electronic devices 80 can also interact with each other by wireless transmission (mobile Internet). Preferably, the electronic device 80 comprises another prompting circuit which can also issue a specific prompt information (for example, by a specific ringtone or vibration mode), and the specific prompt information comprising the first, the second, and the third prompt information as described above.

Alternatively, the electronic device 80 may comprise a prompting circuit (not shown), and the first prompt information may be simultaneously issued by the electronic device 80. The specific implementation process may be, for example, as follows: the processing circuit 20 transmits an analysis result to the electronic device 80 using the communication circuit 70 by means of wireless transmission (Bluetooth, WIFI, mobile Internet, etc.), and the electronic device 80 issues a first prompt information using another prompting circuit in a predetermined manner according to the received analysis result, prompting the user to check whether the medicines are classified incorrectly. The predetermined manner may comprise, for example, a specific ringtone.

Furthermore, the electronic device 80 can also store medication information using a self-contained storage circuit, the medication information comprising at least medication time, the types and the amounts of medicines to be taken every time. Then, the electronic device 80 can receive only the information about the types and the amounts of the medicines sent by the processing circuit 20, and judge, by a self-contained processing circuit, whether the information coincides with the medication information stored in the storage circuit, and then issue a specific prompt information according to the judgement result.

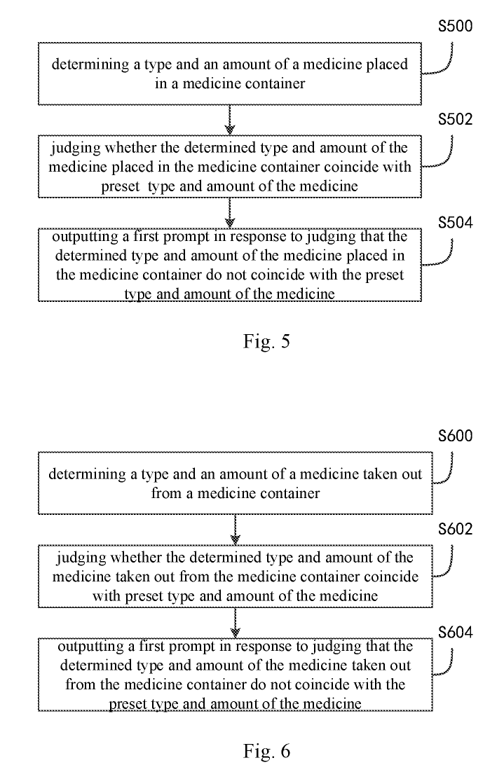

FIG. 5 is a schematic flow diagram of a medicine monitoring method in accordance with some embodiments of the present disclosure. The method is performed, for example, by one or more components of the medicine detecting device and medicine detecting system as shown in FIGS. 1-3. The method comprises the following steps:

step S500: determining a type and an amount of a medicine placed in a medicine container.

step S502: judging whether the determined type and amount of the medicine placed in the medicine container coincide with a preset type and amount of the medicine.

step S504: outputting a first prompt in response to judging that the determined type and amount of the medicine placed in the medicine container do not coincide with the preset type and amount of the medicine. The first prompt is used, for example, to prompt that the type and the amount of the medicine is incorrect and cannot be taken.

In some embodiments, the method may further comprise determining whether at least one subunit contains more than two types of medicines after detecting the types of the medicines in respective subunits of the medicine container. In a case where it is determined that more than two types of the medicines are contained in each of the at least one subunit of the medicine container, a second prompt is output. The second prompt is used, for example, to prompt the patient that the medicines are dispensed incorrectly, and the patient is asked to check and re-dispense the medicines.

FIG. 6 is a schematic flow diagram of a medicine monitoring method in accordance with some embodiments of the present disclosure. The method is performed, for example, by one or more components of the medicine detecting device and medicine detecting system as shown in

FIGS. 1-3. The method comprises the following steps:

S600: determining a type and an amount of a medicine taken out from a medicine container;

S602: judging whether the determined type and amount of the medicine taken out from the medicine container coincide with a preset type and amount of the medicine;

S604: outputting a first prompt in response to judging that the determined type and amount of the medicine taken out from the medicine container do not coincide with the preset type and amount of the medicine. The first prompt is used, for example, to prompt that the type and the amount of the taken-out medicine is incorrect and cannot be taken.

In some embodiments, the method may further comprise determining whether at least one subunit contains more than two types of medicines after detecting the types of the medicines in respective subunits of the medicine container. In a case where it is determined that more than two types of the medicines are contained in each of the at least one subunit of the medicine container, a second prompt is output. The second prompt is used, for example, to prompt the patient that the medicines are dispensed incorrectly, and the patient is asked to check and re-dispense the medicines.

In some embodiments, the method may further comprise determining whether a medicine has been taken out from the medicine container. A third prompt information is issued upon determining that no medicine has been taken out from the medicine container. The third prompt information is used, for example, to remind the user who has forgotten taking medicine to take the medicines.

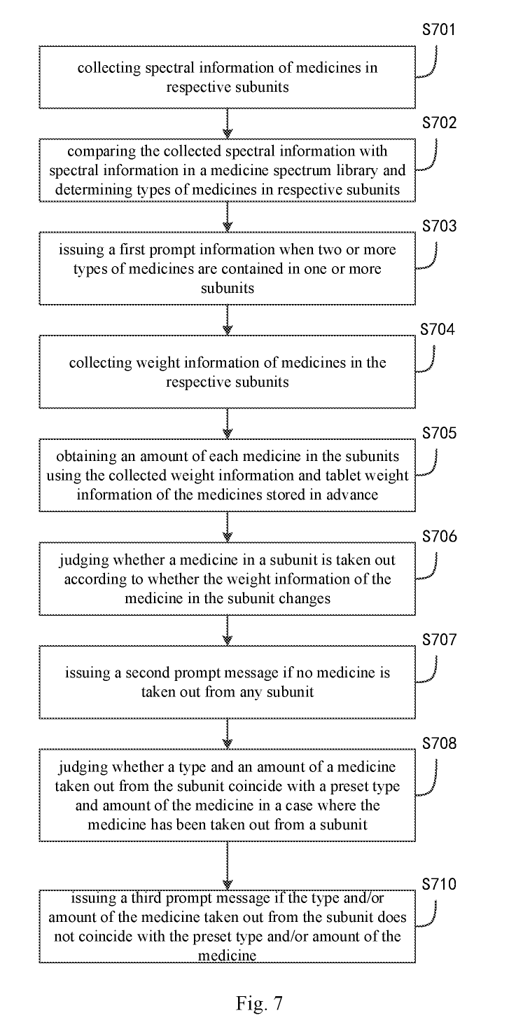

FIG. 7 shows a schematic flow diagram of a medicine monitoring method in accordance with some embodiments of the present disclosure. The method is performed, for example, by one or more components of the medicine detecting device and medicine detecting system as shown in FIGS. 1-3.

As shown in FIG. 7, the method may comprise the following steps:

Step S701: collecting spectral information of medicines in respective subunits;

Step S702: comparing the collected spectral information with spectral information in a medicine spectrum library and determining types of the medicines in the respective subunits.

By the steps S701 and S702, for example, the types of the medicines in the respective subunits of the medicine container can be obtained.

Optionally, the method may further comprise:

Step S703: issuing a first prompt information when two or more types of the medicines are contained in one or more subunits. As described above, the first prompt information may be issued by the prompting circuit 60 as shown in FIG. 2 or may be issued by the prompting circuit of the electronic device 80 as shown in FIG. 3.

The method may comprise:

Step S704: collecting weight information of the medicines in the respective subunits;

S705: obtaining amounts of each type of the medicines in the subunits 11 by using the collected weight information and tablet weight information of the medicines stored in advance.

The amounts of the medicines in the respective subunits can be obtained by step S704 and step S705.

The method may comprise:

Step S705: judging whether a medicine in a subunit is taken out according to whether the weight information of the medicine in the subunit changes; this step is mainly implemented by judging whether the weight information of the medicines in the respective subunits 11 changes.

Step S707: issuing a second prompt information if no medicine is taken out from the subunit. Similar to step S703, the second prompt information may be issued by the prompting circuit 60 located in the medicine monitoring device 200 and/or another prompting circuit of the electronic device 80.

Step S708: in a case where a medicine has been taken out from a subunit, it is judged whether the type and the amount of the medicine taken out from the subunit coincides with the preset type and amount of the medicine.

Step S710: issuing a third prompt information if the type and/or the amount of the medicine taken out from the subunit does not coincide with the preset type and/or amount of the medicine. Similar to step S703, the third prompt information may be issued by the prompting circuit 60 located in the medicine monitoring device 200 and/or another prompting circuit of the electronic device 80.

Compared with the related art, embodiments of the present disclosure have at least one of the following advantages.

Firstly, the spectral characteristics of a medicine in the container can be identified by a Raman analyzer according to different spectrums and can be compared with a medicine spectrum library stored in a storage circuit to obtain the types of the respective medicines, thereby avoiding the problem of misuse of medicines by the patient due to incorrect dispensing of medicines or taking out the wrong medicines.

Secondly, it is also possible to use the weight meter to detect weight information of the medicines in the subunits, remind the patient to take medicine during a medication time period, and ensure that the amounts of the medicines to be taken are consistent with the preset amounts.

With the medicine monitoring device provided by the embodiment of the present disclosure, it can avoid misuse of medicines by the patient caused by placing tablets of different kinds into the same subunit when the tablets are placed in the subunits and can avoid misuse of medicines by the patient caused by taking out incorrect medicines or wrong amounts of the medicines when taking the medicines.

Each embodiment in this description is described in a progressive manner and focuses on differences from other embodiments. For the same or similar parts of the various embodiment, reference can be made to each other. For the system embodiment, since it is substantially similar to the method embodiment, a relatively simple description is given, and reference can be made to the description of the method embodiment for the relevant parts.

The above is a detailed description of the medicine monitoring device, system and method provided by the present disclosure. Specific examples are applied in this specification to elaborate the principles and embodiments of the present disclosure, and the aforementioned descriptions of the embodiments are only used to assist in understanding the method of the present disclosure and its core ideas. For those of ordinary skill in the art, according to the concept of the present disclosure, variations can be made to the embodiments and application scope of the present disclosure. To sum up, the contents of the present disclosure cannot be understood as limitations to the present disclosure.

1. A medicine monitoring device, comprising:
a medicine analyzer configured to detect a type parameter and an amount parameter of a medicine; and
a processing circuit coupled to the medicine analyzer and configured to:
determine a detected type and amount of the medicine based on the detected type parameter and amount parameter of the medicine;
obtain a preset type and amount of the medicine; and
judge whether the detected type and amount of the medicine coincide with the preset type and amount of the medicine.

2. The medicine monitoring device according to claim 1, wherein
the medicine analyzer is configured to detect the type parameter and the amount parameter of the medicine placed in a medicine container.

3. The medicine monitoring device according to claim 1, wherein
the medicine analyzer is configured to detect the type parameter and the amount parameter of the medicine taken out from a medicine container.

4. The medicine monitoring device according to claim 2, wherein the medicine analyzer comprises:
a Raman analyzer comprising a laser device and a laser detecting device, wherein laser light emitted by the laser device is capable of being irradiated onto a surface of the medicine, and the laser detecting device is capable of receiving reflected laser light and obtaining a spectrum of the medicine; and
a weight meter configured to detect a weight of the medicine in the medicine container.

5. The medicine monitoring device according to claim 4, further comprising the medicine container, wherein
the medicine container comprises at least one subunit for holding the medicine,
each subunit is provided with a through hole and is correspondingly provided with the laser device and the laser detecting device, the laser device configured to emit the laser light to be irradiated onto the surface of the medicine in the subunit through the through hole, and the laser detecting device capable of receiving the laser light reflected back through the through hole and obtaining the spectrum of the medicine in the subunit; and
each subunit is correspondingly provided with the weight meter configured to detect the weight of the medicine in the subunit.

6. The medicine monitoring device according to claim 5, wherein the medicine monitoring device further comprises a storage circuit in which a spectrum and a tablet weight of a plurality of medicines are stored in advance.

7. The medicine monitoring device according to claim 6, wherein the processing circuit is further configured to:
determine a type of the medicine in the subunit based on matching between a detected spectrum of the medicine in each subunit and a spectrum of the medicine stored in advance;
calculate an amount of the medicine in the subunit using the detected weight of the medicine in the subunit and the tablet weight of this the determined type of the medicine,
thereby determining the type and the amount of the medicine placed in the medicine container.

8. The medicine monitoring device according to claim 6, wherein in a case where the medicine analyzer is configured to detect the type parameter and the amount parameter of the medicine taken out from the medicine container, the processing circuit is further configured to:
determine a type of the medicine in the subunit based on matching between a spectrum of the medicine in each subunit which is detected before a take-out operation of a user and a spectrum of the medicine stored in advance;
calculate an amount of the determined type of the medicine which has been taken out from the medicine container according to a weight of the determined type of the medicine in the subunit which is detected before the take-out operation of the user, a weight of the determined type of the medicine in the subunit which is detected after the take-out operation of the user and a tablet weight of the determined type of the medicine,
thereby determining the type and the amount of the medicine taken out from the medicine container.

9. The medicine monitoring device according to claim 8, wherein each subunit is configured to allow only one tablet of medicine to be placed, and the processing circuit is further configured to:
determine, in response to detecting a weight of the medicine in the subunit to be zero after the take-out operation of the user, the type and the amount of the medicine taken out from the subunit according to a type of the medicine in the subunit which is detected before the take-out operation of the user,
thereby determining the type and the amount of the medicine taken out from the medicine container.

10. The medicine monitoring device according to claim 1, wherein the processing circuit is configured to output a prompting signal in response to judging that the detected type and amount of the medicine do not coincide with the preset type and amount of the medicine.

11. The medicine monitoring device according to claim 10, further comprising:
a prompting circuit coupled to the processing circuit and configured to present a prompt to a user based on the prompting signal output by the processing circuit.

12. The medicine monitoring device according to claim 10, further comprising a communication circuit coupled to the processing circuit and configured to:
transmit the prompting signal output by the processing circuit to an electronic device in communication with the medicine monitoring device,
receive medication information transmitted from the electronic device, wherein the medication information comprises at least medication time, a type and an amount of the medicine to be taken.

13. A medicine monitoring system, comprising:
the medicine monitoring device according to claim 12, and
an electronic device in communication with the medicine monitoring device,
wherein the medicine monitoring device interacts with the electronic device via the communication circuit.

14. A medicine monitoring method, comprising steps of:
determining a type and an amount of a medicine placed in a medicine container;
judging whether the determined type and amount of the medicine placed in the medicine container coincide with a preset type and amount of the medicine;
outputting a first prompt in response to judging that the determined type and amount of the medicine placed in the medicine container do not coincide with the preset type and amount of the medicine.

15. The medicine monitoring method according to claim 14, further comprising:
outputting a second prompt in response to determining that more than two types of medicines are contained in each of the at least one subunit of the medicine container.

16. A medicine monitoring method, comprising:
determining a type and an amount of a medicine taken out from a medicine container;
judging whether the determined type and amount of the medicine taken out from the medicine container coincide with a preset type and amount of the medicine;
outputting a first prompt in response to judging that the determined type and amount of the medicine taken out from the medicine container do not coincide with the preset type and amount of the medicine.

17. The medicine monitoring method according to claim 16, further comprising:
outputting a second prompt in response to determining that more than two types of medicines are contained in each of the at least one subunit of the medicine container.

18. The medicine monitoring method according to claim 16, further comprising:
outputting a third prompt information in response to determining that no medicine is taken out from the medicine container.

19. The medicine monitoring device according to claim 3, wherein the medicine analyzer comprises:
a Raman analyzer comprising a laser device and a laser detecting device, wherein laser light emitted by the laser device is capable of being irradiated onto a surface of the medicine, and the laser detecting device is capable of receiving reflected laser light and obtaining a spectrum of the medicine; and
a weight meter configured to detect a weight of the medicine in the medicine container.

20. The medicine monitoring device according to claim 11, further comprising a communication circuit coupled to the processing circuit and configured to:
transmit the prompting signal output by the processing circuit to an electronic device in communication with the medicine monitoring device, and
receive medication information transmitted from the electronic device, wherein the medication information comprises at least medication time, a type and an amount of the medicine to be taken.
